(19) United States
(12) Patent Application Publication
(10) Pub. No.: US 2003/0143707 A1
(43) Pub. Date: Jul. 31, 2003

(54) Proteins having DNA repair promoting activity

(75) Inventors: Issay Narumi, Gunma (JP); Katsuya Satoh, Gunma (JP); Suzhen Cui, Gunma (JP); Shigeru Kitayama, Gunma (JP); Hiroshi Watanabe, Gunma (JP)

Correspondence Address: BANNER & WITCOFF, 1001 G STREET N W, SUITE 1100, WASHINGTON, DC, 20001, US

(73) Assignee: Japan Atomic Energy Research Institute, Chiyoda-ku (JP)

(21) Appl. No.: 10/216,862

(22) Filed: Aug. 13, 2002

(30) Foreign Application Priority Data
Aug. 14, 2001 (JP) 2001-246260

(51) Int. Cl. C12N 9/00

(52) U.S. Cl. 435/183; 536/23.1

(57) ABSTRACT

Provided are a protein having DNA repair reaction promoting activity which consists of the amino acid sequence represented by SEQ ID NO: 1 or a modified product of that amino acid sequence, a nucleotide sequence coding for said protein having DNA repair promoting activity, a recombinant DNA having said nucleotide sequence inserted into vector DNA, a transformant containing said recombinant DNA, a process for producing a protein having DNA repair promoting activity comprising the steps of cultivating said transformant in a medium and harvesting a protein having DNA repair promoting activity from the culture, and an antibody binding to said protein.

### BACKGROUND OF THE INVENTION

This invention relates to genes of proteins having DNA repair promoting activity, recombinant vectors containing the genes, transformants containing the recombinant vectors, as well as a process for producing proteins having DNA repair promoting activity using the transformants, and antibodies capable of recognizing the proteins having DNA repair promoting activity.

The radiation resistance of organisms varies greatly with their species. A certain group of microorganisms have the highest resistance to radiation and are collectively referred to as radiation resistant bacteria. A typical example of radiation resistant bacteria is Deinococcus radiodurans whose radiation resistance is about 100 times that of Escherichia coli and about 1,000 times that of human cell. The high radiation resistance of Deinococcus radiodurans is known to be attributable to the ability of that bacterium to repair scission in DNA double strands in an efficient and accurate manner. Therefore, it is believed that by finding DNA repair associated proteins involved in that ability and revealing their functions, the progress of technological development for efficient repair of radiation or otherwise induced difficult-to-repair DNA damage can be promoted to provide a measure for preventing cancer and aging that both occur on account of damaged DNA. In addition, novel DNA repair associated proteins derived from radiation resistant bacteria can contribute to the development of new research reagents for use in DNA manipulation technology and clinical testing and diagnostic agents for typical use in DNA diagnosis.

The genomic DNA of Deinococcus radiodurans has been determined for all of its nucleotide sequences and this bacterium is now known to retain almost all known DNA repair genes which occur extensively in Escherichia coli and other microorganisms (White et al., Science, 286:1571-1577, 1999). However, one can only learn the presence of known repair enzyme genes by searching through genomic sequences and the extremely high radiation resistance of Deinococcus radiodurans cannot be accounted for if it has only the same machinery as that for the DNA repair by less radiation resistant bacteria such as Escherichia coli. Therefore, it may be assumed that the causative gene associated with the outstandingly high DNA repair ability of Deinococcus radiodurans is among the functionally unknown genes that are inherent in this bacterium.

DNA repair genes have been isolated from analyses of mutant Deinococcus radiodurans strains that are deficient in DNA repair ability. For example, a uvrA gene associated with a nucleotide scission repair system was isolated from mutant 3021 strain (Narumi et al., Gene, 198:115-126, 1997); a recA gene associated with a recombination repair system was isolated from mutant rec30 strain (Narumi et al., Mutat. Res., 435:233-243, 1999). A recN gene also associated with a recombination repair system was isolated from mutant KR4128 strain (Funayama et al., Mutat. Res., 435:151-161, 1999). In addition, a recR gene associated with a recombination repair system was isolated from mutant KH5861 strain (Kitayama et al., Mutat. Res., 461:179-187, 2000). However, these are known genes that have also been found in other organisms and no novel gene that is involved in DNA repair has been found from analyses of mutant Deinococcus radiodurans strains that lack DNA repairing ability.

### SUMMARY OF THE INVENTION

The present invention has been accomplished under these circumstances and has as an object providing a novel protein having the activity of promoting the ability of radiation resistant bacteria to repair scission in DNA double strands in an efficient and accurate manner.

Other objects of the invention are to provide a gene for producing the novel protein, a recombinant DNA containing the gene, a process for producing a protein having DNA repair promoting activity using the recombinant DNA, and an antibody capable of recognizing the protein having DNA repair promoting activity.

The present inventors made intensive studies with a view to attaining the stated objects. Based on the analysis of highly radiation sensitive mutant KH3111 strain of Deinococcus radiodurans, the inventors isolated a novel protein having DNA repair promoting activity and successfully defined the DNA nucleotide sequence of its gene. The present invention has been accomplished on the basis of these findings.

To be specific, the first invention of the subject application relates to a protein having DNA repair promoting activity which consists of the amino acid sequence represented by SEQ ID NO: 1. From a different viewpoint, the first invention relates to a protein having DNA repair promoting activity which consists of an amino acid sequence that is identical to the amino acid sequence represented by SEQ ID NO: 1 except that it undergoes deletion, substitution or addition of one or more amino acids. From another different viewpoint, the first invention relates to a protein having DNA repair promoting activity that consists of an amino acid sequence encoded by a nucleotide sequence hybridizable under stringent conditions with a nucleotide sequence complementary to a nucleotide sequence that codes for the amino acid sequence represented by SEQ ID NO: 1 and which is typified by SEQ ID NO: 2. From a further different viewpoint, the first invention relates to a protein having DNA repair promoting activity which consists of an amino acid sequence having at least 60%, preferably at least 70%, more preferably at least 80% and most preferably at least 90%, amino acid sequence homology to the amino acid sequence represented by SEQ ID NO: 1.

The second invention of the subject application relates to a nucleotide sequence that has DNA containing the nucleotide sequence represented by SEQ ID NO: 2 or a nucleotide sequence having degeneracy to it and which codes for a protein having DNA repair promoting activity. From a different viewpoint, the second invention relates to a nucleotide sequence that has DNA containing a nucleotide sequence identical to the nucleotide sequence represented by SEQ ID NO: 2 except that it undergoes deletion, substitution or addition of one or more bases and which codes for a protein having DNA repair promoting activity. From another different viewpoint, the second invention relates to a nucleotide sequence that has DNA containing a nucleotide sequence hybridizable under stringent conditions with a nucleotide sequence complementary to the nucleotide sequence represented by SEQ ID NO: 2 and which codes for a protein having DNA repair promoting activity. From a further different viewpoint, the second invention relates to a nucleotide sequence that has DNA containing a nucleotide sequence having at least 60%, preferably at least 70%, more preferably at least 80% and most preferably at least 90%, amino acid sequence homology to the nucleotide sequence represented by SEQ ID NO: 2 and which codes for a protein having DNA repair promoting activity.

The third invention of the subject application relates to a recombinant vector containing either the gene coding for any one of the proteins having DNA repair promoting activity according to the first invention or the gene according to the second invention.

The fourth invention of the subject application relates to a transformant containing any one of the recombinant vectors according to the third invention.

The fifth invention of the subject application relates to a process for producing a protein having DNA repair promoting activity comprising the steps of cultivating any one of the transformants according to the fourth invention in a medium and harvesting a protein having DNA repair promoting activity from the culture.

The sixth invention of the subject application relates to an antibody that binds to any one of the proteins according to the first invention.

### BRIEF DESCRIPTION OF THE DRAWINGS

FIG. 1 is a schematic representation of the structure of pDC144 and the pZA11 insert;

FIG. 2 shows the result of detecting the pprA gene by Southern hybridization;

FIG. 3 is a schematic representation of the structure of pET3pprAwt;

FIG. 4 shows the Coomassie stain image on SDS-PAGE of a crude product of protein purification;

FIG. 5 shows a gel shift image of linear double-stranded DNA in a crude product of protein purification;

FIG. 6 shows the Coomassie stain images of PprA on SDS-PAGE for various stages in the process of its purification;

FIG. 7 is a photograph showing the DNA binding ability of PprA;

FIG. 8 is a photograph showing the activity of PprA in promoting DNA ligase mediated repair reaction;

FIG. 9 shows the PprA concentration dependency of DNA ligase mediated repair reaction promoting activity for the case of using T4 DNA ligase (FIG. 9A) and for the case of using E. coli DNA ligase (FIG. 9B);

FIG. 10 is a photograph showing the activity of PprA in promoting RecA strand exchange reaction; and

FIG. 11 is a photograph showing the result of analysis by Western blotting of PprA in Deinococcus radiodurans cells.

### DETAILED DESCRIPTION OF THE INVENTION

The present inventors found that a protein having the amino acid sequence represented by SEQ ID NO: 1 had the activity to bind DNA such as an open circular double-stranded DNA with nicks and a linear double-stranded DNA; they also found that the protein enhanced the DNA ligase mediated DNA strand backbone ester binding reaction and the RecA mediated DNA strand exchange reaction. From these results, the present inventors found that the protein of the invention could promote the DNA repair enzyme mediated repair reaction by enhancing the activity of promoting the DNA ligase mediated repair reaction and the activity of promoting the RecA mediated DNA strand exchange reaction. The present invention has been accomplished on the basis of this finding. In the following description, the protein having DNA repair promoting activity which has the amino acid sequence of SEQ ID NO: 1 is designated PprA protein and the gene having the nucleotide sequence of SEQ ID NO: 2 which codes for the PprA protein (SEQ ID NO: 1) is designated pprA gene.

Thus, the PprA protein of the invention is one having DNA repair promoting activity that can bind to an open circular double-stranded DNA having nicks and a linear double-stranded DNA and enhance the activity of promoting DNA ligase mediated repair reaction and the activity of promoting RecA mediated DNA strand exchange reaction, thereby promoting the DNA repair enzyme mediated repair reaction.

The protein of the invention which has DNA repair promoting activity has typically the amino acid sequence represented by SEQ ID NO: 1. The protein may be modified for various purposes including higher affinity for DNA, higher activity, higher throughput or easy purification and promoted passage through organelle lipid membranes and the thus modified proteins are also included in the invention as long as they have DNA repair promoting activity. Hence, the present invention is not limited to the protein having DNA repair promoting activity which consists of the amino acid sequence represented by SEQ ID NO: 1; it also encompasses a protein having DNA repair promoting activity which consists of an amino acid sequence that is identical to the amino acid sequence represented by SEQ ID NO: 1 except that it undergoes deletion, substitution or addition of one or more amino acids; a protein having DNA repair promoting activity that consists of an amino acid sequence encoded by a nucleotide sequence hybridizable under stringent conditions with a nucleotide sequence complementary to a nucleotide sequence that codes for the amino acid sequence represented by SEQ ID NO: 1 and which is typified by SEQ ID NO: 2; a protein having DNA repair promoting activity which consists of an amino acid sequence having at least 60%, preferably at least 70%, more preferably at least 80% and most preferably at least 90%, amino acid sequence homology to the amino acid sequence represented by SEQ ID NO: 1.

The nucleotide sequence of the invention which codes for a protein having DNA repair promoting activity refers typically to a gene coding for a protein having the amino acid sequence of SEQ ID NO: 1 and specific nucleotide sequences include the nucleotide sequence represented by SEQ ID NO: 2 and a nucleotide sequence having degeneracy to it. However, as mentioned above, the proteins of the invention having DNA repair promoting activity include modified products of the PprA protein. Hence, the nucleotide sequence of the invention which codes for a protein having DNA repair promoting activity is not limited to the sequence represented by SEQ ID NO: 2 and a nucleotide sequence having degeneracy to it; also included are genes typified by a nucleotide sequence that has DNA containing a nucleotide sequence identical to the nucleotide sequence represented by SEQ ID NO: 2 except that it undergoes deletion, substitution or addition of one or more bases and which codes for the protein having DNA repair promoting activity; a nucleotide sequence that has DNA containing a nucleotide sequence hybridizable under stringent conditions with a nucleotide sequence complementary to the nucleotide sequence represented by SEQ ID NO: 2 and which codes for the protein having DNA repair promoting activity; and a nucleotide sequence that has DNA containing a nucleotide sequence having at least 60%, preferably at least 70%, more preferably at least 80% and most preferably at least 90%, amino acid sequence homology to the nucleotide sequence represented by SEQ ID NO: 2 and which codes for the protein having DNA repair promoting activity.

Isolation of these genes, preparation of recombinant vectors containing them, preparation of transformants with the use of the recombinant vectors and cultivation of the transformants can be performed by combining known methods such as those described in Sambrook and Russel, Molecular Cloning: A Laboratory Manual, 3rd edition, 2001. For example, genes can be acquired by PCR in which synthetic DNAs of the opposite end portions of the nucleotide sequence represented by SEQ ID NO: 2 are used as probes and the chromosomal DNA is used as a template for amplifying the desired gene. The desired gene can also be acquired by de novo synthesis using more than one synthetic DNA.

“Modified products” of the protein according to the invention include a protein consisting of an amino acid sequence that is identical to the amino acid sequence of the desired protein except that it undergoes deletion, substitution or addition of one or more amino acids; a protein consisting of an amino acid sequence encoded by a nucleotide sequence hybridizable under stringent conditions with a nucleotide sequence complementary to a nucleotide sequence that codes for the amino acid sequence of the desired protein; and a protein consisting of an amino acid sequence having at least 60%, preferably at least 70%, more preferably at least 80% and most preferably at least 90%, amino acid sequence homology to the amino acid sequence of the desired protein.

“Mutants” of the DNA molecule according to the invention include DNA containing a nucleotide sequence having degeneracy to the desired nucleotide sequence; DNA containing a nucleotide sequence that is identical to the desired nucleotide sequence except that it undergoes deletion, substitution or addition of one or more bases; DNA containing a nucleotide sequence hybridizable under stringent conditions with a nucleotide sequence complementary to the desired nucleotide sequence; DNA containing a nucleotide sequence having at least 60%, preferably at least 70%, more preferably at least 80% and most preferably at least 90%, amino acid sequence homology to the desired nucleotide sequence; and DNA containing a nucleotide sequence having degeneracy to these nucleotide sequence.

As described above, the protein having DNA repair promoting activity according to the invention may be a protein having DNA repair promoting activity which consists of an amino acid sequence that is identical to the amino acid sequence of the PprA protein (SEQ ID NO: 1) except that it undergoes deletion, substitution or addition of one or more amino acids. The nucleotide sequence coding for a protein having DNA repair promoting activity according to the invention may be a nucleotide sequence that has DNA containing a nucleotide sequence identical to the nucleotide sequence of the pprA gene (SEQ ID NO: 2) except that it undergoes deletion, substitution or addition of one or more bases and which codes for the protein having DNA repair promoting activity.

The term “one or more” as used herein means preferably 1-20, more preferably 1-10, most preferably 1-5. In the case of protein, “deletion”, “substitution” and “addition” are so effected as to ensure similar properties to those of the PprA protein (SEQ ID NO: 1). In the case of nucleotide sequence, “deletion”, “substitution” and “addition” are so effected in the nucleotide sequence of the pprA gene (SEQ ID NO: 2) as to create a nucleotide sequence coding for a protein having similar properties to those of the PprA protein. Take, for example, the case of “substitution” of amino acids; examples are substitutions of an amino acid by one having similar properties, including the substitution of a certain hydrophobic amino acid by another hydrophobic amino acid, the substitution of a certain hydrophilic amino acid by another hydrophilic amino acid, the substitution of a certain acidic amino acid by another acidic amino acid, and the substitution of a certain basic amino acid by another basic amino acid.

In order to prepare proteins having the above-defined “deletion”, “substitution” or “addition” or nucleotide sequences having the “deletion”, “substitution” or “addition” defined above, one may employ various methods known in the art of the invention, as exemplified by site directed mutagenesis, random mutagenesis employing mutagenic treatment and errors in PCR amplification, and cassette mutagenesis.

As described above, the protein having DNA repair promoting activity according to the invention may be a protein having DNA repair promoting activity that consists of an amino acid sequence encoded by a nucleotide sequence hybridizable under stringent conditions with a nucleotide sequence complementary to a nucleotide sequence that codes for the amino acid sequence of the PprA protein (SEQ ID NO: 1) and which is typified by the pprA gene (SEQ ID NO: 2). The nucleotide sequence coding for a protein having DNA repair promoting activity according to the invention may be a nucleotide sequence that has DNA containing a nucleotide sequence hybridizable under stringent conditions with a nucleotide sequence complementary to the nucleotide sequence of the pprA gene (SEQ ID NO: 2) and which codes for the protein having DNA repair promoting activity.

The stringent conditions as used herein refer to those conditions under which the desired nucleotide sequence is capable of specific hybridization with a nucleotide sequence (e.g. SEQ ID NO: 2) that codes for the PprA protein (SEQ ID NO: 1) or a nucleotide sequence that has degeneracy to it. The hybridizing conditions are determined in consideration of temperature, ion concentration and other conditions; it is generally known that stringency increases with increasing temperature and lower ion concentration. Suitable stringent conditions can be set by the skilled artisan on the basis of known information, for example, in accordance with Sambrook and Russel, Molecular Cloning: Laboratory Manual, 3rd edition, 2001. A specific example of stringent conditions is hybridization conditions described by 6×SSC, 5× Denhard's, 0.1% SDS and 25° C.-68° C. A more preferred range of hybridization temperature is from 45° C. to 68° C. (without formamide) or from 25° C. to 50° C. (in 50% formamide).

As described above, the protein having DNA repair promoting activity according to the invention may be a protein having DNA repair promoting activity which consists of an amino acid sequence having at least 60%, preferably at least 70%, more preferably at least 80% and most preferably at least 90%, amino acid sequence homology to the amino acid sequence of the PprA protein (SEQ ID NO: 1). The nucleotide sequence coding for a protein having DNA repair promoting activity according to the invention may be a gene typified by a nucleotide sequence that has DNA containing a nucleotide sequence having at least 60%, preferably at least 70%, more preferably at least 80% and most preferably at least 90%, amino acid sequence homology to the nucleotide sequence of the pprA gene (SEQ ID NO: 2) and which codes for the protein having DNA repair promoting activity.

In the present invention, the sequence homology of amino acid or nucleotide sequences may be determined by visual inspection or mathematical calculation. Alternatively, the sequence homology between two protein sequences may be determined on the basis of the algorithm of Needleman and Wunsch (J. Mol. Biol., 48:443-453, 1970) and by comparing the sequence information using the GAP computer program available from the University of Wisconsin Genetic Computer Group (UWGCG). Preferred default parameters in the GAP program include: (1) scoring matrix, blosum62, as described in Henikoff and Henikoff, Proc. Natl. Acad. Sci. USA, 89:10915-10919, 1992, (2) weighting of 12 gaps, (3) weighting of 4 gap lengths, and (4) no penalty on terminal gaps.

In the present invention, the sequence homology of amino acid or nucleotide sequences may also be analyzed by using other sequence comparison programs commonly adopted by the skilled artisan. To mention just one example, the BLAST program described in Altschul et al. (Nucl. Acid. Res. 25:3389-3402, 1997) may be used to effect comparison with the sequence information of interest for determination purposes. Specifically, in the case of analyzing nucleotide sequences, the Nucleotide BLAST (BLASTN) program may be used to enter the Query nucleotide sequence which is then checked against nucleotide sequence databases such as GenBank, EMBL and DDBJ. In the case of analyzing amino acid sequences, the Protein BLAST (BLASTP) program may be used to enter the Query amino acid sequence which is then checked against amino acid sequence databases such as GenBank CDS, PDB, SwissProt and PIR. These programs can be accessed on the Internet from the web site of National Center for Biotechnology Information (NCBI) or DNA Data Bank of Japan (DDBJ). Those sites offer detailed information about the conditions (parameters) for searching on homology by the BLAST programs. Although the settings of these conditions may partly be modified as appropriate, search is usually performed with default values. Other sequence comparison programs familiar to the skilled artisan may also be employed.

The amino acid sequence of the PprA protein (SEQ ID NO: 1) according to the invention and the nucleotide sequence of the pprA gene (SEQ ID NO: 2) coding for the PprA protein were subjected to sequence homology search on the basis of the above-described sequence comparison. As it turned out, the PprA protein and pprA gene according to the invention had no significant sequence homology to any one of the proteins and genes known in the art.

The pprA gene of the invention may be cloned on the basis of the disclosure in Narumi et al. (Gene, 198:115-126, 1997), Funayama et al. (Mutat. Res., 435:151-161, 1999) or Narumi et al. (Mutat. Res., 435:233-243, 1999) by introducing the DNA from a radiation-resistant wild-type strain into a highly radiation-sensitive mutant strain of Deinococcus radiodurans and selecting clones capable of reverting the mutant to acquire resistance to mutagenic stimulus. Specifically, the pprA gene may be cloned by the following methods:

(1) The DNA in a cosmid library derived from a radiation-resistant wild-type strain is introduced into a highly radiation-sensitive mutant strain of Deinococcus radiodurans and cosmid clones capable of reverting the mutant to acquire resistance to mutagenic stimulus are selected;

(2) In order to further focus on the site at which the desired gene exists in the cosmid clones obtained in (1), each of the cosmid clones having the insert digested with restriction enzymes is inserted into a suitable vector and using the thus prepared vector, the mutant is again transformed and subclones capable of reverting the mutant to acquire resistance to mutagenic stimulus are selected;

(3) In order to focus further down on the desired gene which exists in the subclones obtained in (2), nested deletion plasmids are prepared from those subclones and subjected to another on mutant strain transformation experiment, whereby the DNA region having the activity of reverting the mutant to acquire resistance to mutagenic stimulus is specified and cloned;

(4) The sequence of the insert DNA cloned in above (3) is determined.

The recombinant vector of the invention may be obtained by incorporating the thus cloned pprA gene into a vector DNA in accordance with common techniques such as those described in Sambrook and Russel, Molecular Cloning: A Laboratory Manual, 3rd edition (2001). Vectors that can be used include but are not limited to pUC19 (Takara Shuzo), pBluescript II KS(+) (Stratagene) and pET3a (Novegen). Specifically, the multiple cloning site on the vector is cleaved with one or two appropriate restriction enzymes; in a separate step, the opposite ends of the cloned pprA gene are cleaved said with one or two appropriate restriction enzymes that can form the same cohesive or blunt end as the fragment cleaved with the first mentioned one or two appropriate restriction enzymes; the open circular vector having a cohesive or blunt end at opposite termini and the pprA gene fragment prepared as described above are ligated to construct the recombinant vector of the invention.

The thus prepared recombinant vector may be introduced into host cells such as E. coli, Bacilus substilis, yeast and mammalian cell by known transformation techniques such as the ones described in detail by Sambrook and Russel (Molecular Cloning: A Laboratory Manual, 3rd edition (2001)), Hardin (Cloning, Gene Expression and Protein Purification: Experimental Procedures and Process Rationale (2001)), and Brown (Essential Molecular Biology: A Practical Approach, Vol. 1, 2nd edition (2001)), whereby transformants are produced that are capable of expressing DNA repair promoting activity. The other transformation techniques described in any publications may of course be employed and the host cells that can be used are by no means limited to the examples mentioned above. Specifically, in order to prepare transformants, the recombinant vector obtained in the manner described above is transferred into the host cell using transformation techniques known in the relevant art. Specific transformation techniques known in the relevant art include but are not limited to the method of using bacteriophages, the method of using liposomes, the particle gun technique, electroporation and the calcium chloride method.

Known methods for producing the protein having DNA repair promoting activity using the thus prepared transformants are exemplified by but not limited to the ones described in detail by Simon (Protein Purification Techniques: A Practical Approach, The Practical Approach Series, 244, 2nd edition (2001)), Simon (Protein Purification Applications: A Practical Approach, The Practical Approach Series, 245, 2nd edition (2001)), and Hardin (Cloning, Gene Expression and Protein Purification: Experimental Procedures and Process Rationale (2001)). Specifically, the transformant may be cultivated under culture conditions that are suitable for the production of the protein having DNA repair promoting ability and which are also suitable for the growth of the specific host used; the harvested cells are sonicated or otherwise disrupted, and centrifuged to produce the desired protein. If necessary, the centrifugal supernatant may be purified on commercial media including an ion-exchange resin, a gel filtration carrier and an affinity resin. The thus produced protein having DNA repair promoting activity may subsequently be treated by known methods such as digestion with trypsin, pepsin, etc. and used as a domain peptide having the desired activity.

The present invention further relates to an antibody that recognizes the protein having DNA repair promoting activity. This antibody of the invention can be acquired by known methods, typically described in detail by Delves (Antibody Production: Essential Techniques (1997)), Haward and Bethell (Basic Methods in Antibody Production and Characterization (2000)) and Kontermann and Dubel (Antibody Engineering: Springer Lab Manual (2001)) using the protein having DNA repair promoting activity which has been purified by the aforementioned methods or fragments of such protein. The antibody of the invention may be polyclonal or a monoclonal antibody. If a suitable animal is immunized with the protein having DNA repair promoting activity or a fragment thereof and the serum is collected from the immunized animal, the desired polyclonal antibody can be obtained from the serum. Animals to be immunized generally include but are not limited to rabbit, sheep, goat, guinea pig and mouse. To obtain the monoclonal antibody, the following procedure may be taken: the antibody producing cells are recovered from the animal immunized with the above-described protein having DNA repair promoting activity or a fragment thereof; the recovered antibody producing cells are subjected to cell fusion with a suitable fusion partner such as myeloma cell, whereby hybridoma cells are obtained; then, clones producing an antibody having the required activity are cultivated for subcloning in a medium suitable for their growth; the subcloned hybridoma cells are cultivated under suitable conditions and the desired monoclonal antibody can be acquired from the conditioned medium. The antibody can also be produced by allowing the thus obtained hybridoma cells to grow intraperitoneally in mammals. Preferred animals to be immunized include mouse, nude mouse, rat and chicken. The thus obtained antibody may be purified by common isolation and purification methods including centrifugation, dialysis, salting out with ammonium sulfate and the like, ion-exchange chromatography, gel filtration and affinity chromatography.

The antibody of the invention thus obtained can be used for various purposes including purification of the protein having DNA repair promoting activity, detection of it and inhibition of its activity. The antibody of the invention can be used after it is converted to an F(ab′)₂ fragment or an Fab′ fragment by known methods. If the antibody of the invention is to be used to detect the protein having DNA repair promoting activity, it may preliminarily be labelled with a radioisotope (e.g. ³⁵S or ³H) or an enzyme (e.g. horseradish peroxidase) or a suitable affinity ligand (e.g. avidin-biotin).

The PprA protein of the invention has the activity of binding to open circular double-stranded DNA and linear double-stranded DNA. By making use of this activity of the PprA protein, one can perform enhanced purification of DNA or mRNA.

For example, a closed circular double-stranded plasmid DNA of high purity is required as a template for efficient performance of DNA sequencing or preparation of a deficient library. However, if plasmid DNA is extracted from E. coli or other organisms by existing methods, cleavage may have occurred to the DNA strands during the extraction process and may contain a mixture of an open circular double-stranded DNA component and a linear double-stranded DNA component. Such open circular double-stranded DNA and linear double-stranded DNA can be removed from the plasmid DNA by a suitable technique such as passing it through a column in which the PprA protein of the invention has been immobilized. Therefore, the PprA protein of the invention can be incorporated in a kit for extracting the closed circular double-stranded plasmid DNA.

The PprA protein of the invention binds specifically to DNA, so in order to remove genomic DNA in mRNA that can cause false positivity in RT-PCR reaction, it may be passed through the PprA immobilizing column mentioned above, whereupon high-purity mRNA is obtained. Therefore, the PprA protein of the invention can be incorporated in a kit for extracting high-purity mRNA.

In still another application, there may be prepared a column in which the anti-PprA antibody of the invention is immobilized and the reaction for binding between the antibody and PprA may be used to acquire the above-described high-purity closed circular double-stranded plasmid DNA and mRNA.

Imported beef and fruit are usually sterilized by exposure to radiation and it has become important to develop a convenient method for checking irradiated food. This is another area where the PprA protein and the anti-PprA antibody can be put to use. For example, by combining them with the comet assay used as one of the methods for detecting irradiated food, the cleavage of DNA strand can be quantitated more accurately. If immunohistological testing and other methods are used, the amount of DNA cleavage in the irradiated food can be compared with that for the unirradiated control to provide a more convenient way to locate the irradiated food. Therefore, the PprA protein of the invention, optionally combined with the anti-PprA antibody, can be incorporated in a kit for detecting and/or quantitating DNA cleavage.

The PprA protein of the invention has the activity of promoting the DNA ligase mediated DNA strand backbone ester binding reaction. DNA ligase is one of the enzymes most frequently used in gene manipulation technology. Therefore, a research reagent capable of efficient ligase reaction could be provided by combining DNA ligase with the PprA protein. Heat-resistant DNA ligase is used to detect SNP (single nucleotide polymorphism) by ligation-dependent PCR. Therefore, modified products of the PprA protein that have both DNA ligase promoting activity and heat resistance are useful in the development of an efficient ligation-dependent PCR technique for gene diagnosis.

The PprA protein of the invention has the activity of promoting the DNA strand exchange reaction in RecA protein, namely, its homologous recombination activity. As described by Ferrin and Camerini-Otero (Proc. Natl. Acad. Sci. USA, 95:2152-2157, 1997) and Ferrin (DNA Repair Protocols: Procaryotic Systems, Methods in Molecular Biology, Vol. 152:135-147, 2000), the homologous recombination activity of RecA is applied in molecular biological DNA manipulation technology for various purposes such as protecting a specific DNA region from the digestion with restriction enzymes, RARE digestion by restriction enzymes through inhibited methylation and RecA-assisted cloning. Further, as described in U.S. Pat. No. 4,888,274, the homologous recombination reaction of RecA is also applied to realize convenient screening by capturing target clones from a plasmid cDNA library. By combining the PprA protein of the invention with RecA, the DNA strand exchange reaction or homologous recombination reaction which are mediated by pairing between homologous sequences of RecA can be allowed to proceed more efficiently.

The following examples are provided to further illustrate the present invention but are in no way to be taken as limiting.

### EXAMPLES

### Example 1

### Cloning of pprA Gene

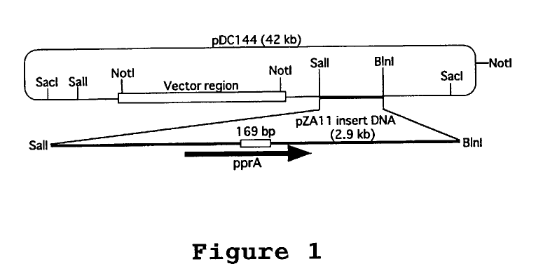

The DNA of a cosmid library from a radiation-resistant wild-type strain (Narumi et al., Gene, 198:115-126, 1997) was introduced into a highly radiation-sensitive Deinococcus radiodurans mutant KH3111 strain (Kitayama et al., J. Bacteriol., 155:1200-1207, 1983) and clones capable of reverting the mutant KH3111 strain to acquire resistance to mitomycin C (Kyowa Medex Co. Ltd.) were selected; pDC144 (42 kb) was obtained from the clones (FIG. 1). To give a brief description of the method for preparing the cosmid library from a radiation-resistant wild-type strain, genomic DNA of wild-type strain KD8301 was partially digested with restriction enzyme MboI (Takara Shuzo) and the digested genomic DNA was packaged into a SuperCos 1 cosmid vector (Stratagene) which in turn was incorporated into an E. coli XL1-Blue MR strain (Stratagene) (Narumi et al., Gene, 198:115-126, 1997).

Spontaneous transformation of Deinococcus radiodurans was effected in accordance with the method of Kitayama et al. (Kitayama et al., J. Bacteriol., 155:1200-1207, 1983). Stated briefly, bacteria which is cultured in a liquid media was mixed with DNA in the presence of calcium chloride and cultivated in a liquid medium, followed by plating on a selective agar medium containing mitomycin C.

The mitomycin C resistance conferred by the DNA of pDC144 indicates the presence of a mutagenicity gene in the insert in the clone. Hence, in order to focus on the site at which the desired gene existed, the pDC144 insert was digested with restriction enzymes and subcloned into pUC19 (Takara Shuzo) and using the subclones, the mutant KH3111 strain was again transformed to select clones capable of reverting the mutant to acquire mitomycin C resistance. As a result, a plasmid was obtained that contained a SalI-BlnI fragment (2.9 kb) from pDC144 and the plasmid was named pZA11 (FIG. 1).

In order to focus further down on the site at which the desired gene existed in pZA11, a nested deletion plasmid of pZA11 was prepared using a Kilo-Sequencing Deletion kit (Takara Shuzo) and subjected to an experiment on transformation of the mutant KH3111 strain. As a result, a DNA region having the activity of reverting the mutant KH3111 strain to acquire mitomycin C resistance was found to be 169 bp long (FIG. 1).

The sequence of the pZA11 insert DNA was determined using 377 DNA Sequencing System (Applied Biosystems); an 855-bp open reading frame (SEQ ID NO: 2) was found in a DNA region including the aforementioned 169-bp region.

The amino acid sequence (SEQ ID NO: 1) deduced from the sequence of this open reading frame was not homologous to any one of the proteins of known sequences and, hence, was a novel protein. The gene capable of reverting the DNA repair deficiency in mutant KH3111 strain was named pprA and the novel DNA repair associated protein encoded by this gene was named PprA.

### Example 2

### Detection of pprA Gene

In this example, the pprA gene was detected by Southern hybridization. To this end, two Deinococcus radiodurans strains were used; one was Deinococcus radiodurans strain R₁ (ATCC 13939) which was the parent of strain KD8301 used to isolate the pprA gene and which had 5 pigment components, and the other was Deinococcus radiodurans strain Sark (ATCC 35073) having 6 pigment components, one of which was shared by strain R₁ but the other five were not. Deinococcus radiodurans strain Sark was of the same species as strain R₁ but established as a different strain.

Restriction enzyme digested genomic DNA was prepared from Deinococcus radiodurans strains R₁ and Sark in accordance with the method of Kikuchi et al. (FEMS Microbiol. Lett., 174:151-157, 1999) and subjected to pulsed-field gel electrophoresis, then to Southern blot analysis in accordance with the method described by Sambrook and Russel in Molecular Cloning: A Laboratory Manual, 3rd edition (2001). The specific procedure was as follows.

Cells embedded in 1% low-melting agarose GB (Nippon Gene) were treated at 37° C. for 24 hours in 1 mL of a buffer (10 mM Tris-HCl, pH 8.0, 40 mM EDTA, 50 mM sucrose, 0.1% Triton X-100) containing 1 mg/mL lysozyme (Sigma). The subsequent treatment was within the same buffer containing 1 mg/mL of Proteinase K (Qiagen) and continued for 24 hours at 50° C. Thereafter, digestion with 30 U of restriction enzyme NotI (Takara Shuzo) was effected at 37° C. for 24 hours in 100 μL of a restriction enzyme reaction buffer (50 mM Tris-HCl, pH 7.5, 10 mM MgCl₂, 1 mM dithiothreitol, 100 mM NaCl, 0.01% fetal bovine serum albumin, 0.01% Triton X-100). Thereafter, using Geneline I (Beckman), pulsed-field gel electrophoresis was performed at a constant current of 150 mA with a switch time of 20 seconds in a 1% agarose HS (Nippon Gene) gel for 16 hours using a TAFE buffer (10 mM Tris-HCl, 0.5 mM EDTA, 4.4 mM acetic acid), whereby restriction enzyme digested genomic DNA was resolved.

After electrophoresis, the restriction enzyme digested genomic DNA resolved in the gel was transferred to a nylon membrane (Roche Diagnostics) for 24 hours in an alkali buffer (0.4 N NaOH, 1 M NaCl) in accordance with the usual manner. Thereafter, the membrane was heat treated at 80° C. for 2 hours so that the restriction enzyme digested genomic DNA was immobilized on the membrane.

A DNA fragment having the structural gene sequence (nucleotide sequences 1-855) of the pprA gene isolated in Example 1 was digoxigenin-labeled with DIG DNA labelling kit (Roche Diagnostics). The digoxigenin-labeled DNA fragment was used as a probe and hybridized with the restriction enzyme digested DNA immobilizing membrane under stringent conditions. Specifically, the hybridization reaction was performed at 68° C. for 18 hours using a hybridizing solution (5×SSC, 1% blocking solution, 0.1% N-lauryl sarcosine, 0.02% SDS). Thereafter, the reacted membrane was washed twice, each for 5 minutes, with a buffer of high ionic strength (2×SSC, 0.1% SDS) at room temperature, then washed twice, each for 15 minutes, with a buffer of low ionic strength (0.1×SSC, 0.1% SDS) at 68° C. The labelling of the probe was thereafter detected with DIG DNA detection kit (Roche Diagnostics).

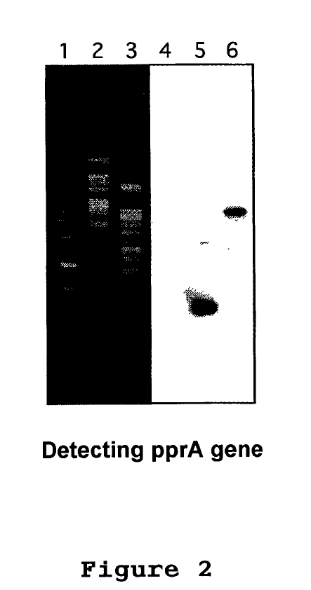

The result is shown in FIG. 2. The respective lanes were loaded with the following: molecular weight marker (lane 1); Deinococcus radiodurans strain R₁ (lane 2); Deinococcus radiodurans strain Sark (lane 3); molecular weight marker (lane 4); Deinococcus radiodurans strain R₁ (lane 5); and Deinococcus radiodurans strain Sark (lane 6). Lanes 1-3 show the electrophoretic gels stained with ethidium bromide and lanes 4-6 show the results of Southern hybridization.

As is clear from FIG. 2, digesting the genomic DNAs of Deinococcus radiodurans strain R₁ and Deinococcus radiodurans strain Sark with NotI gave DNA fragments of entirely different patterns. This shows that strains R₁ and Sark which are two bacteria belonging to the genus Deinococcus should more correctly be classified in different species or subspecies. Although the pprA gene of the invention was isolated from strain R₁, the pprA gene was also detected from strain Sark in lane 6.

### Example 3

### Preparation of PprA Overproducing Plasmid

In order to realize overproduction of the novel DNA repair associated protein PprA by Deinococcus radiodurans, the following two oligonucleotide primers were designed.

[Primer 1]
5′-GGGCATAATA AAGGCCATAT GGCAAGGGCT AAAGC-3′
[Primer 2]
5′-TTTTGGATCC TCAGCTCTCG CGCAGGCCGT GC-3′

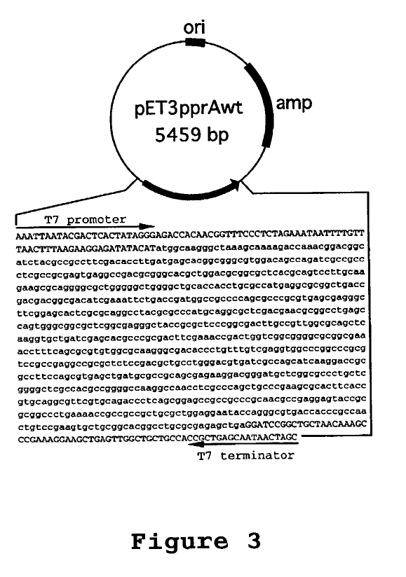

With pZA11 used as a template, PCR was performed using the above defined sense primer and antisense primer. The PCR product was digested with restriction enzymes NdeI and BamHI and subcloned into the NdeI-BamHI site of E. coli expression vector pET3a (Novagen); the subclone was named pET3 pprAwt (FIG. 3).

### Example 4

### Preparation of Transformants and Crude Product of Protein Purification

In order to obtain a wild-type protein, E. coli BL21 (DE3) pLysS (Novagen) was transformed with pET3 pprAwt plasmid (FIG. 3). Individual transformants were cultivated in an LB medium (BD Bioscience) containing ampicillin and chloramphenicol; when the absorbance at a wavelength of 600 nm reached 0.6, IPTG (isopropyl-β-D-thiogalactopyranoside, Takara Shuzo) was added to induce protein at a final concentration of 0.4 mM.

After cultivation for an additional 3 hours, centrifugation was effected to produce a cell pellet of transformants. The cell pellet was suspended in a lysis buffer (20 mM Tris-HCl, pH 8.0, 2 mM EDTA, 1 mM PMSF) and disrupted by sonication. After centrifugating at 8,000 rpm for 30 minutes, the supernatant was recovered to obtain a crude product of protein purification.

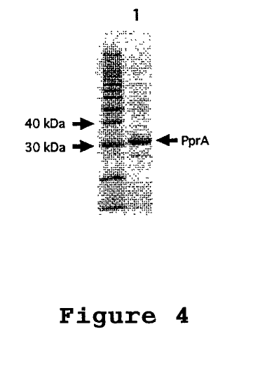

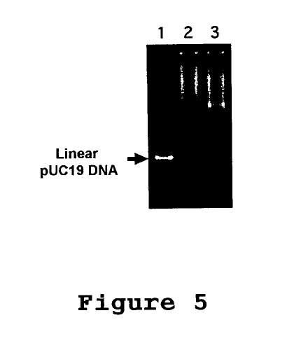

The expression of protein was confirmed by SDS-PAGE (FIG. 4 in which lane 1 is for the culture of E. coli BL21 (DE3) pLysS pET3pprAwt). The expressed protein had a molecular weight of 31.6 kDa which was in agreement with the value deduced from the sequence represented by SEQ ID NO:1. The crude protein was mixed with the linear double-stranded DNA of pUC19 (Takara Shuzo) and the mixture was incubated in a buffer (10 mM Tris-HCl, pH 7.5, 10 mM MgCl₂) for 30 minutes; upon agarose gel electrophoresis, the wild-type protein was found to have DNA binding ability (FIG. 5, in which lane 1 is for the linear double-stranded DNA of pUC19, lane 2 for the crude product of protein purification from E. coli BL21 (DE3) pLysS pET3pprAwt, and lane 3 for the addition of the linear double-stranded pUC19 DNA to lane 2).

### Example 5

### Purification of Wild-Type PprA

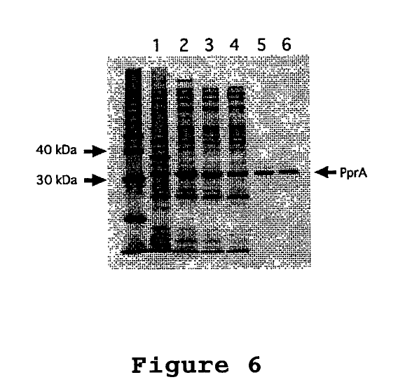

The crude protein obtained in Example 4 was purified by polyamine treatment, salting out with ammonium sulfate, chromatography on DEAE Sepharose CL-6B column (Amersham Pharmacia Biotech), filtration through Sephacryl S-300 gel (Amersham Pharmacia Biotech) and chromatography on mono Q column (Amersham Pharmacia Biotech) to recover the wild-type protein (FIG. 6, in which lane 1 is for the supernatant of a centrifuged solution of sonicated cells, lane 2 for the product of polyamine treatment, lane 3 for the product of ammonium sulfate treatment, lane 4 for the product from chromatography on DEAE Sepharose CL-6B column, lane 5 for the product of filtration through Sephacryl S-300 gel, and lane 6 for the product of chromatography on mono Q column).

### Example 6

### Evaluating the Properties of PprA

(1) The N-terminal Amino Acid Sequence

The protein purified by the method of Example 4 was subjected to SDS-PAGE and transferred to a membrane filter (Milliopore); thereafter, the N-terminal amino acid sequence of the protein was determined with PSQ-1 protein sequencer (Shimadzu). As it turned out, all of the 12 amino acids that could be analyzed were in agreement with the sequence represented by SEQ ID NO: 1.

(2) DNA Binding Ability

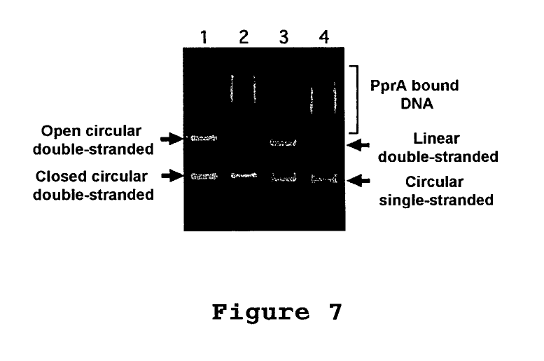

The protein purified by the method of Example 4 was closely evaluated for DNA binding ability. To begin with, a closed circular double-stranded, an open circular double-stranded, a circular single-stranded and a linear double-stranded sample of φX174 DNA (New England Biolabs) were prepared. These DNA samples and the protein purified by the method of Example 4 were reacted in a buffer (10 mM Tris-HCl, pH 7.5, 10 mM MgCl₂) for 10 minutes and then subjected to agarose gel electrophoresis for gel shift assay (FIG. 7, in which lane 1 is for the closed and open circular double-stranded samples of φX174 DNA, lane 2 for the addition of PprA to lane 1, lane 3 for the linear double-stranded and circular single-stranded samples of φX174 DNA, and lane 4 for the addition of PprA to lane 3). Specific binding to PprA was only found in the open circular and linear double-stranded DNA samples, suggesting that PprA would participate in DNA repair through recognizing a breakage damage to the DNA strands and binding to it.

(3) The Activity of Promoting the DNA Ligase Mediated Repair Reaction

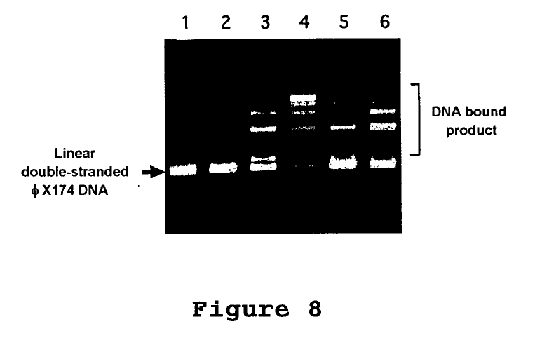

The effect of PprA on the DNA ligase mediated DNA strand backbone ester binding reaction was evaluated. The linear double-stranded sample of φX174 DNA prepared in above (2) was used as the substrate DNA. The substrate DNA was mixed with T4 DNA ligase (Promega) or E. coli DNA ligase (Takara Shuzo) and with the protein purified by the method of Example 4; following 10-minute incubation, DNA was purified by phenol treatment and ethanol precipitation and subjected to agarose gel electrophoresis. As it turned out, the addition of PprA increased the yield of the DNA bound product by up to 55.3% and this verified the DNA ligase mediated repair reaction promoting activity of PprA (FIG. 8, in which lane 1 is for the linear double-stranded sample of φX174 DNA, lane 2 for the addition of PprA to lane 1, lane 3 for the addition of T4 DNA ligase to lane 1, lane 4 for the addition of PprA to lane 3, lane 5 for the addition of E. coli DNA ligase to lane 1, and lane 6 for the addition of PprA to lane 5).

(4) PprA Concentration Dependency of the DNA Ligase Mediated Repair Reaction Promoting Activity

An experiment was conducted as described in above (3) and the ratio of the DNA bound product was determined. In addition, the concentration of PprA was measured.

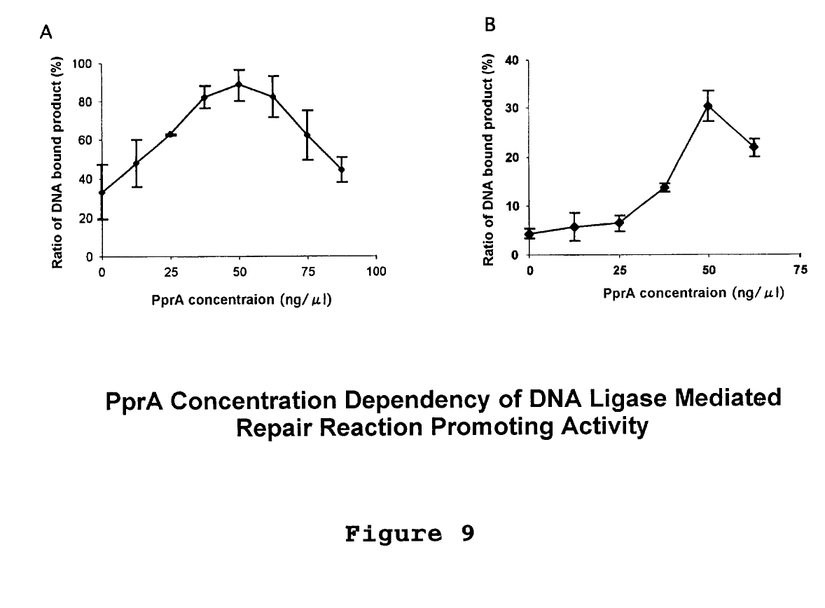

Whether T4 DNA ligase was used (case A) or E. coli DNA ligase was used (case B), the promoting effect of the PprA protein was maximum when it was added in an amount of 50 ng/μL to 7.5 ng/μL of the substrate DNA (FIGS. 9A and 9B). In the case of T4 DNA ligase, the ratio of the DNA bound product was 32.9% in the absence of the PprA protein but increased to 88.2% when it was added in an amount of 50 ng/μL. In the case of E. coli DNA ligase, the ratio of the DNA bound product was 4.3% in the absence of PprA but increased to 30.3% when it was added in an amount of 50 ng/μL (FIGS. 9A and 9B).

(5) The Activity of Promoting RecA Mediated DNA Strand Exchange Reaction

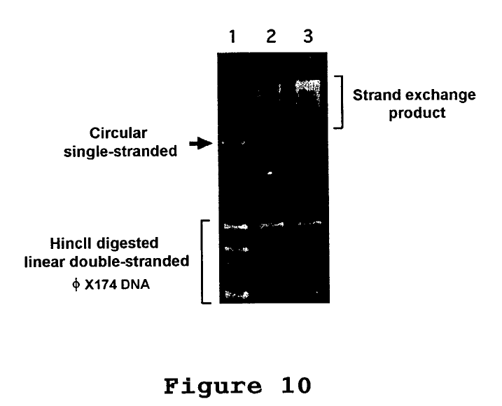

The effect of PprA on the recombinant protein RecA mediated DNA strand exchange reaction was evaluated. E. coli RecA (Promega) was used as the recombinant protein RecA. Two samples were prepared for the substrate DNA; one was a closed circular double-stranded sample of φX174 DNA that was digested with restriction enzyme HincII (Takara Shuzo) and the other was a circular single-stranded sample of φX174 DNA. The DNA strand exchange reaction was performed by the method of Muller et al. (Muller et al., Methods in Mol. Biol., 30, 413-423, 1994). The addition of PprA increased the yield of the strand exchange product by up to 20%, verifying the RecA mediated DNA strand exchange reaction promoting activity of PprA (FIG. 10, in which lane 1 is for the substrate DNA alone, lane 2 for the addition of E. coli RecA to lane 1, and lane 3 for the addition of PprA to lane 2). This result led the present inventors to conclude that PprA was a protein capable of promoting the reaction for repair by a DNA repair enzyme.

### Example 7

### Preparing Polyclonal Antibody Recognizing the PprA Protein Having DNA Repair Promoting Activity

(1) Preparation of Polyclonal Antibody

A pure protein prepared by the method of Example 4 was used as an immunogen. A rabbit was subcutaneously immunized with 100 μL of an emulsion of this antigen in Freund's oily adjuvant. Once half a month, an emulsion of the antigen in an equal amount of Freund's oily adjuvant was applied as a booster (for a total of 6 times). One month later, a whole blood sample was taken from the rabbit. The blood was centrifuged to separate serum which was immobilized by heat inactivation and stored at −80° C. after addition of 0.05% NaN₃.

(2) Specificity of Polyclonal Antibody

Specificity of the antibody was evaluated by detecting PprA in Deinococcus radiodurans cells. To begin with, a pprA gene disruptant strain was prepared by chloramphenicol resistance gene cassette introduction in accordance with the method of Funayama et al. (Funayama et al., Mutat. Res., 435:151-161, 1999); the disruptant strain was designated XN1.

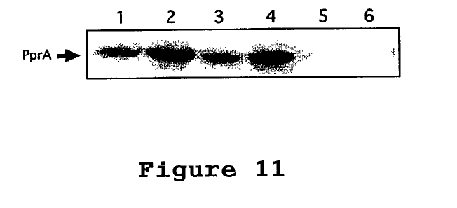

Then, a wild-type strain of Deinococcus radiodurans, the mutant KH311 strain and the disruptant XN1 strain were each exposed to 2 kGy of gamma-rays and subjected to culture using shaker for 2 hours. Thereafter, the harvested cells were disrupted with glass beads and centrifuged to prepare a protein extract. The extract was subjected to SDS-PAGE and the separated protein was transferred to a membrane filter (Millipore); thereafter, Western blot analysis was performed using the antibody prepared in above (1). The PprA protein was detected in the wild-type strain and the mutant KH3111 strain but not in the pprA gene disruptant strain (FIG. 11, in which lane 1 is for the wild-type strain not exposed to gamma-rays, lane 2 for the wild-type strain exposed to gamma-rays, lane 3 for the mutant KH3111 strain not exposed to gamma-rays, lane 4 for the mutant KH3111 strain exposed to gamma-rays, lane 5 for the gene disruptant XN1 strain not exposed to gamma-rays, and lane 6 for the gene disruptant XN1 strain exposed to gamma-rays). The result demonstrated the specificity of the prepared antibody. Use of the antibody showed that PprA was radiation inducible.

### INDUSTRIAL APPLICABILITY

The present invention provides a novel protein having DNA repair promoting activity. Known repair enzymes such as DNA ligase, DNA polymerase and nucleases are used as research reagents in gene manipulation technology and also as agents for clinical testing and diagnosis of DNA. Therefore, the protein having the activity of promoting the reaction involving known repair enzymes can contribute to the development of more efficient research reagents, clinical testing and diagnostic agents, etc. Since the protein of the invention which has DNA repair promoting activity recognizes a scission in DNA strands and binds specifically to it, this characteristic of the invention can be utilized to develop a new reagent for use in detecting a scission site in DNA strands or in other DNA manipulation techniques. If desired, the protein of the invention which has DNA repair promoting activity may be combined with an antibody that recognizes it and this can either control the reaction system involving the above-mentioned research or diagnostic reagents or improve the sensitivity in detection by them. The antibody can also be used to capture proteins that will bind to the protein PprA having DNA repair promoting activity, hopefully contributing to unraveling the mechanism of DNA repair. The antibody can also be used to search for proteins having DNA repair promoting activity from organisms other than Deinococcus radiodurans.

What is claimed is:

1. A protein having DNA repair promoting activity which consists of the amino acid sequence represented by SEQ ID NO: 1 or an amino acid sequence that is identical to the amino acid sequence represented by SEQ ID NO: 1 except that it undergoes deletion, substitution or addition of one or more amino acids.

2. A protein having DNA repair promoting activity that consists of the amino acid sequence represented by SEQ ID NO: 1 or an amino acid sequence encoded by a nucleotide sequence hybridizable under stringent conditions with a nucleotide sequence complementary to a nucleotide sequence that codes for the amino acid sequence represented by SEQ ID NO: 1.

3. A protein having DNA repair promoting activity which consists of the amino acid sequence represented by SEQ ID NO: 1 or an amino acid sequence having at least 60% amino acid sequence homology to the amino acid sequence represented by SEQ ID NO: 1.

4. DNA that has DNA consisting of the nucleotide sequence represented by SEQ ID NO: 2 or a nucleotide sequence identical to the nucleotide sequence represented by SEQ ID NO: 2 except that it undergoes deletion, substitution or addition of one or more bases and which codes for a protein having DNA repair promoting activity.

5. DNA that has DNA consisting of the nucleotide sequence represented by SEQ ID NO: 2 or a nucleotide sequence hybridizable under stringent conditions with a nucleotide sequence complementary to the nucleotide sequence represented by SEQ ID NO: 2 and which codes for a protein having DNA repair promoting activity.

6. DNA that has DNA consisting of a nucleotide sequence having at least 60% amino acid sequence homology to the nucleotide sequence represented by SEQ ID NO: 2 and which codes for a protein having DNA repair promoting activity.

7. A recombinant vector containing DNA having a nucleotide sequence coding for the protein according to any one of claims 1-3.

8. A recombinant vector containing the DNA according to any one of claims 4-6.

9. A transformant containing the recombinant vector containing DNA having a nucleotide sequence coding for the protein according to any one of claims 1-3 or the DNA according to any one of claims 4-6.

10. A process for producing a protein having DNA repair promoting activity comprising the steps of cultivating the transformant according to claim 9 in a medium and harvesting a protein having DNA repair promoting activity from the culture.

11. An antibody that binds to the protein according to any one of claims 1-3.


SEQUENCE LISTING (4 sequences)

SEQ ID NO: 1 - protein, 284 aa, Deinococcus radiodurans
  source (1)..(284): Amino acid sequence of DNA repair promoting protein of Deinococcus radiodurans, strain KD8301.
MARAKAKDQTDGIYAAFDTLMSTAGVDSQIAALAASEADAGTLDAALTQSLQEAQGRWGLGLHHLRHEARLTDDGDIEILTDGRPSARVSEGFGALAQAYAPMQALDERGLSQWAALGEGYRAPGDLPLAQLKVLIEHARDFETDWSAGRGETFQRVWRKGDTLFVEVARPASAEAALSDAAWDVIASIKDRAFQRELMRRSEKDGMLGALLGARHAGAKANLAQLPEAHFTVQAFVQTLSGAAARNAEEYRAALKTAAAALEEYQGVTTRQLSEVLRHGLRES

SEQ ID NO: 2 - DNA, 855 bp, Deinococcus radiodurans
  source (1)..(855): Nucleotide sequence of DNA repair promoting protein of Deinococcus radiodurans, strain KD8301.
atggcaagggctaaagcaaaagaccaaacggacggcatctacgccgccttcgacaccttgatgagcacggcgggcgtggacagccagatcgccgccctcgccgcgagtgaggccgacgcgggcacgctggacgcggcgctcacgcagtccttgcaagaagcgcaggggcgctgggggctggggctgcaccacctgcgccatgaggcgcggctgaccgacgacggcgacatcgaaattctgaccgatggccgccccagcgcccgcgtgagcgagggcttcggagcactcgcgcaggcctacgcgcccatgcaggcgctcgacgaacgcggcctgagccagtgggcggcgctcggcgagggctaccgcgctcccggcgacttgccgttggcgcagctcaaggtgctgatcgagcacgcccgcgacttcgaaaccgactggtcggcggggcgcggcgaaacctttcagcgcgtgtggcgcaagggcgacaccctgtttgtcgaggtggcccggcccgcgtccgccgaggccgcgctctccgacgctgcctgggacgtgatcgccagcatcaaggaccgcgccttccagcgtgagctgatgcgccgcagcgagaaggacgggatgctcggcgccctgctcggggctcgccacgccggggccaaggccaacctcgcccagctgcccgaagcgcacttcaccgtgcaggcgttcgtgcagaccctcagcggagccgccgcccgcaacgccgaggagtaccgcgcggccctgaaaaccgccgccgctgcgctggaggaataccagggcgtgaccacccgccaactgtccgaagtgctgcggcacggcctgcgcgagagctga

SEQ ID NO: 3 - DNA, 35 bp, Artificial
  Sense primer for amplifying pprA gene.
gggcataataaaggccatatggcaagggctaaagc

SEQ ID NO: 4 - DNA, 32 bp, Artificial
  Antisense primer for amplifying pprA gene.
ttttggatcctcagctctcgcgcaggccgtgc